(19) United States
(12) Patent Application Publication
(10) Pub. No.: US 2011/0137209 A1
(43) Pub. Date: Jun. 9, 2011

(54) MICROPHONE ARRAYS FOR LISTENING TO INTERNAL ORGANS OF THE BODY

(76) Inventors: ROSA R. LAHIJI, Shaker Heights, OH (US); Mehran Mehregany, San Diego, CA (US)

(21) Appl. No.: 12/917,848

(22) Filed: Nov. 2, 2010

Related U.S. Application Data
(60) Provisional application No. 61/258,082, filed on Nov. 4, 2009.

(51) Int. Cl. A61B 7/00

(52) U.S. Cl. 600/586

(57) ABSTRACT

An electronic device is provided for receiving sounds from a body. A microphone array receives the sounds. An analysis system optionally provides for directional control, such as by providing virtual focusing and beam steering. Body sounds are preferably de-convolved. In certain embodiments, a plurality of buffer structures are located in cavities in a patch adjacent the microphones to provide for improved sound pick-up. In certain embodiments, at least two of microphones are spaced at least 2 centimeters apart. Preferably, wireless transmission circuitry sends information relating to the sounds in the body, and optionally receives information, such as control or status information. Target selection and acquisition systems provide for the effective capture of multiple sounds from the body, even when the device is adhered to the body by the user, that is, not a skilled physician.

### RELATED APPLICATION DATA

This application claims priority to and benefit of U.S. Provisional Application Ser. No. 61/258,082, filed Nov. 4, 2009, entitled “Microphone Arrays for Listening to Internal Organs of the Body”, the content of which is incorporated by reference herein in its entirety as if fully set forth herein.

### FIELD OF THE INVENTION

The present invention relates to methods, apparatus and systems for listening to internal organs of a body. More particularly, it relates to arrays of microphones for the improved detecting of sounds in internal organs of a body, especially in a wearable configuration adapted for wireless communication with a remote site.

### BACKGROUND OF THE INVENTION

Detection and analysis of sounds from the internal organs of the body is often a first step in assessment of a patient's condition. For example, accurate auscultation of heart and lung sounds is used routinely for detection of abnormalities in their functions. A stethoscope is the device most commonly used by physicians for this purpose. Modern stethoscopes incorporate electronic features and capabilities for recording and transmitting the internal organ sounds. Existing devices often utilize a single microphone for recording of the body's internal organ sounds and perform post-filtering and electronic processing to eliminate the noise. S. Mandal, L. Turicchia, R. Sarpeshkar, “A Battery-Free Tag for Wireless Monitoring of Heart Sounds”, Sixth International Workshop on Wearable and Implantable Body Sensor Networks, pp. 201-206, June 2009.

In general, more sophisticated noise-canceling techniques involve two microphones, for example in applications such as (i) capturing and amplifying the sound of a speaker in a large conference room or (ii) in some modern laptops combining signals received from two microphones where the main sensor is mounted closest to the intended source and the second is positioned farther away to pick up environmental sounds that are subtracted from the main sensor's signal. Reported stethoscope work uses similar techniques to capture the intended signal along with the ambient noise. Y.-W. Bai, C.-H. Yeh, “Design and implementation of a remote embedded DSP stethoscope with a method for judging heart murmur”, IEEE Instrumentation and Measurement Technology Conference, pp. 1580-1585, May, 2009. Chan US 2008/0013747 proposes using a MEMS array for noise cancellation, where a first microphone picks up ambient noise, and the second picks up heart or lung sounds.

Other techniques involve adaptive noise cancellation using multi-microphones. See, e.g., Y.-W. Bai, C.-L. Lu, “The embedded digital stethoscope uses the adaptive noise cancellation filter and the type I Chebyshev IIR bandpass filter to reduce the noise of the heart sound”, IEEE Proceedings of international workshop on Enterprise networking and Computing in Healthcare Industry (HEALTHCOM), pp. 278-281, June 2005. After the signals have been combined properly, sounds other than the intended source are greatly reduced. In a mechanical stereo-scopy stethoscope device, Berk et al. U.S. Pat. No. 7,516,814 proposes a mechanical approach using constructive interference of sound waves.

Sensors that convert audible sound into an electronic signal are commonly known as microphones. High performance, digital MEMS microphone are available in ultra miniature form factor (e.g., approaching 1 mm on a side and slightly lesser thickness in packaged form), at very low power consumption. These microphones (and generally other small, inexpensive microphones) have an omni-directional performance (FIG. 1), resulting in the same performance along all the incident angles of sound.

Directivity of the microphone is an important feature to eliminate the surrounding noise and produce the sound of the internal organ of interest, e.g., heart/lung sound. Often times enlarging the size of a single sensing element (either a microphone or other sensors such as piezoelectric devices) leads to more directive characteristics. See, e.g., C. A. Balanis, “Antenna Theory”, J. Wiley, 2005. This approach is used in implementing the Littmann® electronic stethoscopes (3100 and 3200) (see FIG. 2). In this product environmental noise is further reduced by using a built-in gap in the stethoscope head's sidewalls for mechanically filtering the ambient noise.

FIG. 3(a) shows the four different recognized positions to hear the sound of heart functions. See, e.g., Bai and Yeh, above. FIG. 3(b) shows the Bai and Yeh proposed ideal location for the two separated stethoscope heads in order to cancel noise using digital signal processing techniques (DSP) and to distinguish the heart sound from the lung sound. As seen in FIG. 3(b), there needs to be a specific distance between the two stethoscope heads for successful performance, which complicates the use of this device as patients vary in size.

In yet other applications of microphones, modern hearing aid devices use source localization and beam-forming techniques to track the sound source for better hearing experience. S. Chowdhury, M. Ahmadi, W. C. Miller, “Design of a MEMS acoustical beam forming sensor microarray”, IEEE Sensors Journal, Vol. 2, Issue 6, pp. 617-627, December 2002. Because of the size constraint of placing the device in the ear canal, the array is effectively a point source.

There is a wide variation in acoustical properties of commercially-available electronic stethoscopes arising from either the choice of the sensor or the mechanical design. However, producing a high quality, noise-free sound output, covering the entire 20 Hz to 2 KHz spectrum, has proved to be a challenge. A pure heart/lung sound for example, when captured electronically, can not only be recorded but also transmitted (wirelessly) to a hands-free hearing piece or to a healthcare provider (server) for further analysis or for archiving in electronic records. Benefits of such electronic recording, analysis, transmission, and archiving of body sounds is compelling in many settings, including ambulatory, home, office, hospital, and trauma care to name a few.

Finally, in a wireless environment, the microphone will often need to be operated without physician guidance of the device. Accordingly, the skilled physical manipulation and position of the stethoscope provided by the physician is not available in such systems. Further, to promote patient acceptance and comfort, it is desirable to have a small, compact device, as opposed to a bulky vest type monitoring system.

According, an improved system is required.

### SUMMARY OF THE INVENTION

An array of miniature microphones based preferably on microelectromechanical systems (MEMS) technology provides for directional, high quality and low-noise recording of sounds from the body's internal organs. The microphone array architecture enables a recording device with electronic spatial scanning, virtual focusing, noise rejection, and deconvolution of different sounds. This auscultation device is optionally in the form of a traditional stethoscope head or as a wearable adhesive patch, and can communicate wirelessly with a gateway device (on or in the vicinity of the body) or to a network of backend servers. Applications include, for example, for physician and self-administered, as-needed and continuous monitoring of heart and lung sounds, among other internal sounds of the body. Array architecture provides redundancy, ensuring functionality even if a microphone element fails.

The system preferably includes a microphone array comprised of elements that are preferably ultra small and very low cost (e.g., MEMS microphones), which are used for electronic spatial scanning, virtual focusing, noise rejection, and deconvolution of different sounds. The array is implemented as a linear array or as a non-linear array, and may be planar or may be three dimensional. A microphone array structure is preferably disposed adjacent a housing. The microphone array includes a plurality of individual microphones, which are preferably held in an array configuration by a support. The outputs of the microphones in this embodiment are connected to conductors to conduct the microphone signals to the further circuitry for processing, preferably including, but not limited to amplifiers, phase shifters and signal processing units, preferably digital signal processing units (DSPs). Processing may be in the analog domain, or the digital domain, or both. The output of the analysis system is then provided to the transmit/receive module Tx/Rx, which is either coupled wirelessly through an inductive link (passive telemetry) to a device in vicinity of the body or through a miniaturized antenna to a network for archiving, such as in backend servers.

Through the analysis system, the system may perform one or more of the following functions: electronic spatial scanning, virtual focusing, noise rejection, feature extraction and de-convolution of different sounds. By using a DSP chip and combining the outputs from a multi-microphone array in any desired fashion, a single virtually-focused microphone with steerable gaze is achieved.

According to one embodiment, an electronic scope is provided for receiving sounds in a body. The scope preferably includes a microphone array structure, the structure including at least a first microphone, the first microphone including an electrical output corresponding to sounds in the body, a second microphone, the second microphone including an electrical output corresponding to sounds in the body, and a support. The support is connected to at least the first and second microphones to hold them in an array configuration. An analysis system is provided which includes a directional processing system coupled to receive the output from the microphone array system, and signal processing circuitry to analyze the sounds in the body. The signal processing circuitry preferably includes digital signal processing. Finally, a wireless transmission circuitry sends and optionally receives information relating to the sounds in the body or other control functions.

In yet another embodiment, an electronic device is provided for receiving sounds in a body, including a plurality of microphones, a corresponding plurality of buffer structures, and a patch structure. The patch structure preferably includes at least a patient side surface and an opposed side surface. The patch has a plurality of cavities, the cavities being adapted to receive the buffer structures and to maintain the buffer structures adjacent the plurality of microphones. In certain embodiments, at least two of microphones are spaced at least 2 centimeters apart. The device electronics include signal processing circuitry to analyze the sounds in the body. Preferably, wireless transmission circuitry sends information relating to the sounds, and optionally receives information, such as control or status information.

The microphone array system of the present invention permits the beam gaze to be virtually steerable so as to focus on desired sounds from specific organs of the body. Target selection may be either direct, such as when input locally by the user or medical professional, or remotely, such as from a remote server, or indirect such as when the various organs are sequentially scanned for sounds.

Accordingly, it is an object of these inventions to provide a wearable scope, such as a wearable stethoscope, which provides for the effective capture of sounds in the body.

It is yet a further object of these inventions to provide a microphone array which provides for spatial scanning, or virtual focusing, on sounds within the body.

### BRIEF DESCRIPTION OF THE DRAWINGS

FIG. 1 shows the prior art depicting the pattern of an omni-directional microphone showing its gain to the sound coming from different angles (θ) with respect to its central axis.

FIG. 2 shows the prior art depicting the directionality of a stethoscope and ambient noise reduction.

FIG. 3(a) shows the prior art known locations for hearing the sounds from four valves function of the heart and FIG. 3(b) location for noise cancellation techniques using two microphones.

FIG. 4A shows a perspective view of the patient side surface of a disk shaped microphone array.

FIG. 4B shows a perspective view of the patient side surface of an annular shaped microphone array.

FIG. 4C shows a perspective view of the patient side surface of a semi-spherical 3-dimensional shaped microphone array.

FIGS. 5A and 5B show a perspective view of the patient side and opposed side, respectively, of a patch type sound capturing device, including the microphones and circuit topology.

FIGS. 6A and 6B show plan and perspective views, respectively, of the external portion of a compound patch sound capturing device.

FIGS. 6C and 6D show plan and perspective views, respectively, of the patient side and opposed side of a patient disposed portion of the compound patch of FIGS. 6A through 6D, combined.

FIGS. 7A and 7B show a plan and cross-sectional view of the patient side of a patch structure.

FIG. 8 shows a block diagram of the components of the scope.

FIG. 9 is a perspective view of a wireless patch and associated processing or input/output devices.

FIG. 10 shows the steerable gaze of an array with virtual focusing in various directions of θ₁, θ₂, and θ₃.

FIG. 11 shows directivity and gain patterns (y-z plane) of a two-element microphone array when d=0.4λ compared to a single microphone, wherein N is the number of microphones.

FIG. 12 shows the architecture of a planar microphone array in x-z plane, with dₓ spacing along x-axis and d_{z} spacing along the z-axis between the elements.

FIG. 13 shows performance of a linear array in y-z plane when d=0.2λ and N=1, 2, 3 and 4.

FIG. 14 shows performance of a three-element linear array in y-z plane when the distance between the elements is varied from 0.1λ to 0.4λ.

FIG. 15 shows steering the beam in y-z plane by changing the electronic phase φ from 0° to 60° in a three-element array with spacing of 0.4λ.

FIG. 16 shows different spatial beam configurations formed by different arrays by changing the spacing and number of microphones, as well as progressive electronic phase shifts between the elements.

FIG. 17 is a flowchart of the operational process flow.

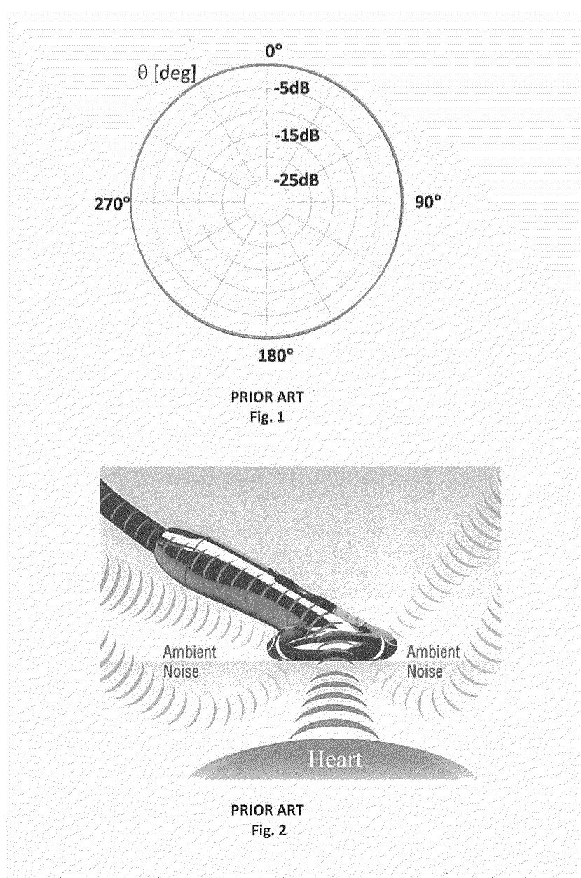

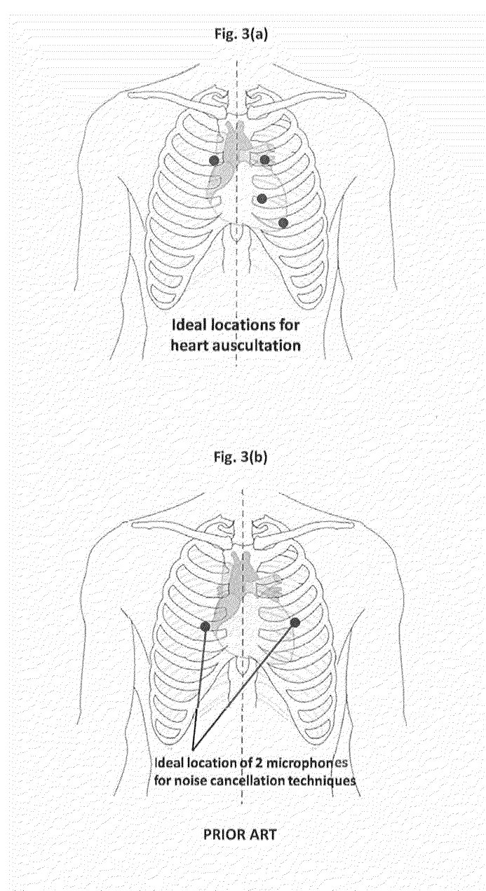

### DETAILED DESCRIPTION OF THE INVENTION

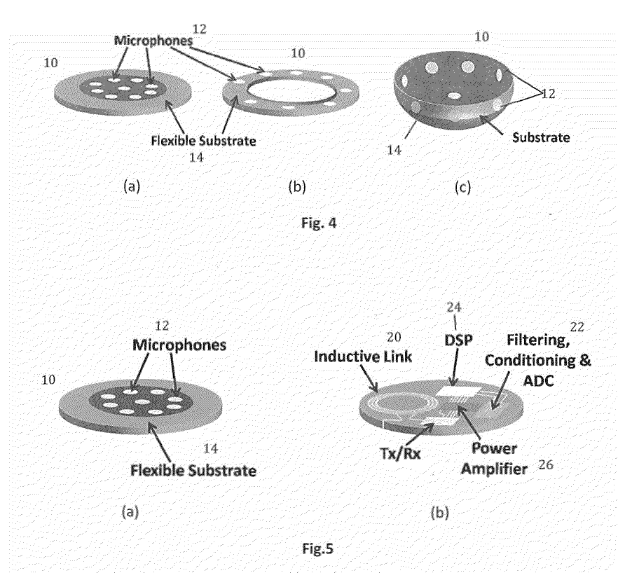

FIGS. 4A, 4B and 4C show three schematic representations of implementations of the apparatus and system of these inventions. FIG. 4A shows a generally planar, circular arrangement. FIG. 4B shows a generally annular arrangement, having a center opening. FIG. 4C shows a three dimensional, semi-spherical, arrangement. The microphone array 10 includes a plurality of individual microphones 12. The microphones 12 are in turn supported by or disposed upon or adjacent a support or substrate 14. As shown by way of example, in FIG. 4A there are 9 microphones 12 arrayed in a circular manner around a central microphone 12. As shown in FIG. 4B, eight microphones 12 are disposed around the annular substrate 14. As shown in FIG. 4C, there are 7 microphones 12 disposed around the periphery of the substrate with additional microphones also located on the support 14. Optionally the support 14 is flexible, such as to permit an intimate contact with the body to optimize sound transmission. Further, a composite or multi-component support may be utilized. The location and placement of the microphones in FIGS. 4 A, B and C are not meant to be limitative. The placement, array formation and orientation of the microphones 12 is treated in detail, particularly with reference to FIGS. 10 through 16, and the accompanying description, below. The microphones 12 each include an output, the outputs in this embodiment being connected to conductors (vias or wires or leads) to conduct the microphone signals to the further circuitry for processing.

FIGS. 5 A and B show a simplified front end circuitry for a microphone array, and further processing for transmission of the sounds by wireless communication. FIG. 5A shows a perspective view of the system described, for example, with reference to FIG. 4A, but the description applies to all microphone array structures 10 described herein. FIG. 5B shows the reverse side of FIG. 5A and included the analysis system 20. Generally, the output of the microphones 12 is passed through conductors, vias, wires, leads, or wireless transmission to the input system 22. Optionally, the input system 22 may include filtering and conditioning functionality. Additionally, in the event that the signals from the microphones 12 are analog, and the system is to operate in the digital domain, an analog to digital converter (ADC) is utilized. Optionally, a preamplifier, especially a low noise preamplifier, may be utilized, as necessary. In yet another variant, one or more phase shifters may be included in the initial processing system 22 as desired. The analysis system 20 preferably includes a digital signal processor (DSP) 24 for analyzing the signals from the various microphones. The DSP is coupled to receive the output of the initial processing system 22. A power amplifier is preferably coupled to the DSP 24. Any particular architecture for implementation of these functionalities may be selected as would readily be appreciated by those skilled in the art.

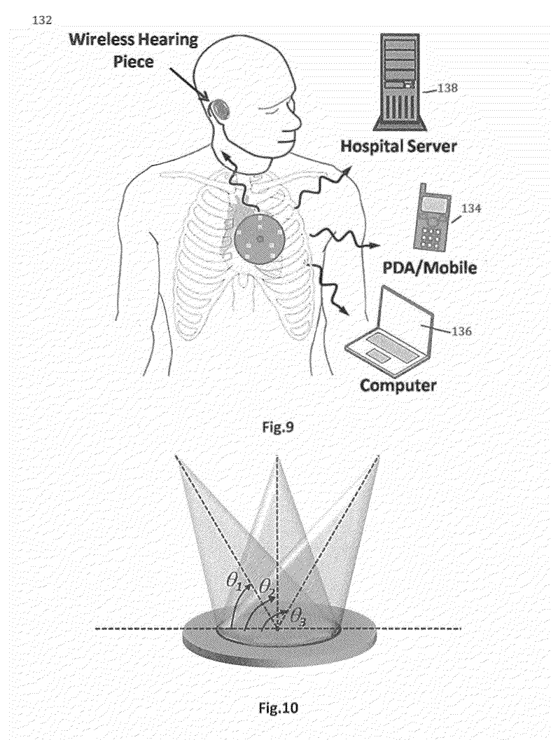

The output of the analysis system 20 is then provided to wireless transmission circuitry 28. The wireless transmission circuitry includes at least a transmit capability, and optionally includes a receive capability as well. The wireless transmission circuitry 28 is either coupled to an inductive link 30 in vicinity of the body (passive telemetry) or a miniaturized antenna (not shown) for communication and archiving in backend servers through a network (See, e.g., FIG. 9).

Through the analysis system 20, the system may perform one or more of the following functions: electronic spatial scanning, virtual focusing, noise rejection, and deconvolution of different sounds. By using a DSP chip and combining the outputs from a multi-microphone array in any desired fashion, a single virtually-focused microphone with steerable gaze is achieved.

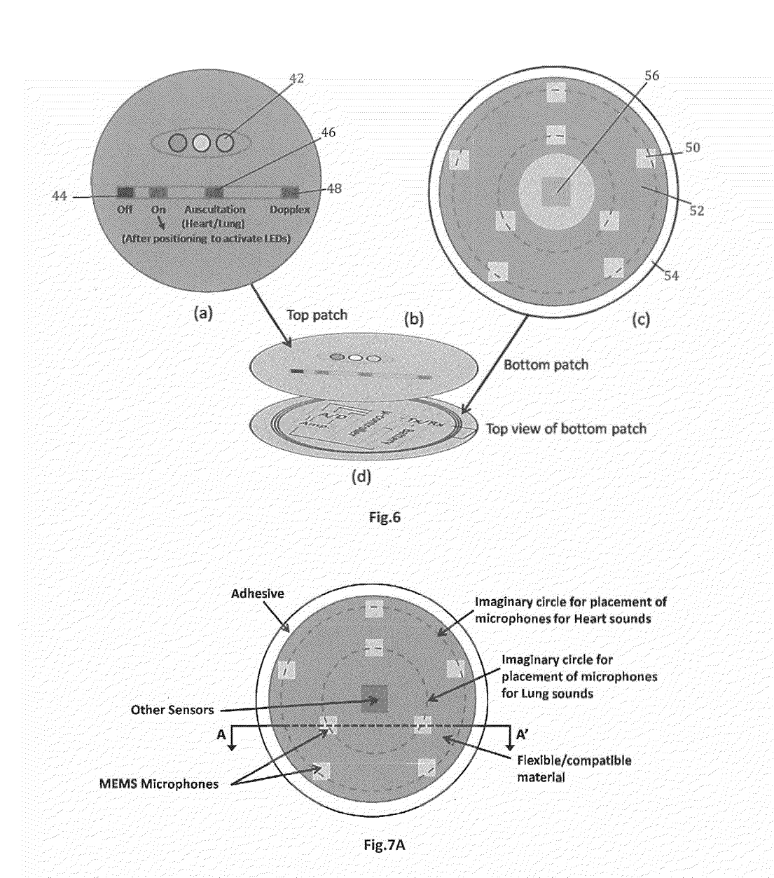

FIGS. 6 A and B show plan and perspective views, respectively, of one embodiment of the sensor array systems. FIGS. 6C and 6D show the patient side and opposed side, respectively, of a patch adapted to join with the patch of FIGS. 6A and B. As shown in FIGS. 6 A and B, the external portion of the compound patch may include functionality for input and output. By way of example, in order to further assist the user, signaling devices such as colorful LEDs 42 may be incorporated into the auscultation piece to indicate when the user has placed it optimally, i.e., where the desired signal levels are strong. The signaling devices 42 may be used for other output or patient advising information, such as to indicate battery level or the proper orientation of the device in the event the device has an asymmetry. Various color coding may be used, such as red to indicate a weak signal level, yellow to indicate a medium-to-moderate signal, and green to indicate a strong signal level. Optionally, and on/off switch 44 may be provided. The visible portion 40 may optionally include an auscultation function 46 which may be used by the patient or physician to indicate to the unit the desired sound to acquire, or may serve as an output indicator to indicate the sound currently being captured. Doppler functionality 48 may be displayed to show the Doppler mode has been invoked.

FIG. 6C shows the patient side of the device, including microphones 50 arrayed adjacent the substrate 52. Optionally an adhesive 54 may be disposed to aid in the attachment or affixing of the device to the patient. As shown, an optional additional sensor 56 may be utilized. Optional additional sensors include, but are not limited to, temperature sensors, accelerometers, piezoelectric sensors, ECG electrodes and gyroscopes. As shown in FIG. 6D, the output from the microphones 50 is coupled or transmitted to, optionally, an amplifier 62, and further coupled to an analog to digital (A/D) converter 64, if processing is to occur in the digital domain. A power source, optionally a battery 66, may be included. Wireless transmission circuitry 68 is shown as having both transmit and receive functionality (Tx/Rx). As before, the particular components and architecture to implement the desired functionality may be in any mode or form of implementation as is readily known to those skilled in the art.

In the structure of FIGS. 6 A through D, the electronic components optionally may be located or sandwiched between the opposed side of the patient patch and the inner side of the external patch. Alternately, the electronics may be formed on a flexible electronics support, such as a flexible printed circuit board. The components that interface with the patient, e.g., microphones 50 and additional sensors 52, may be formed in one region, and the electronics formed external to that region. The flexible electronic support may be folded or wrapped around such that the components that interface with the patient are in one direction, and the other electronics are directed away from the patient. In this way, electronic connections, such as circuit traces, may connect from the components that interface with the patient, to the electronics for analysis without needing to pass through the patch.

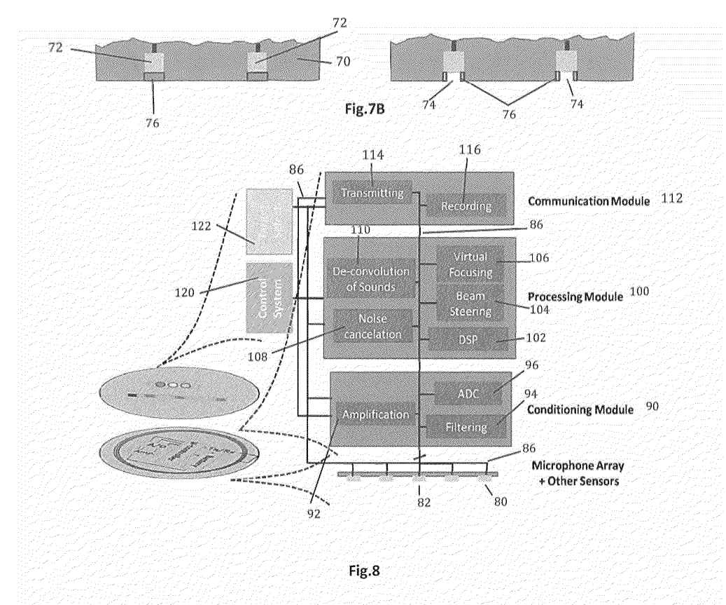

FIG. 7A shows the structure of FIG. 6C, but further includes cut line A-A′ to show the cut line for FIG. 7B. In FIG. 7B, the substrate 70 is shown in cross-section. Microphones 72 are disposed in or on the substrate 70 to be located adjacent a cavity 74. The cavity 74 is in turn adapted to contain a buffer structure 76. The buffer structure serves to better couple sounds from the body to the microphones 72. Buffer structures 76 may include, but are not limited to, rubber, metal, and metal alloys. The buffer structures preferably are adapted to be retained in the cavities 74, in a sound transmitting relationship with the microphones 72. The cavity height can be as low as 2 millimeters in size (diameter and or depth). As shown in the left hand of FIG. 7B, the buffer materials fills the entire cavity, and is preferably a non-metallic material, such as rubber. The right hand of FIG. 7B shows the cavity with buffer sidewalls, thereby leaving an air gap within the cavity adjacent at least a portion of the microphone. In this embodiment, the buffer material may be selected from the full array of buffer materials, above.

The microphones 50 may optionally be placed in a configuration to optimize the detection of sounds from desired organs. In one exemplary embodiment shown in FIG. 6C and FIG. 7A, three inner microphones are arranged in an imaginary circle for detection of lung sounds, whereas the three outer microphones are arranged in an imaginary circle for detection of heart sounds.

In one implementation, a plurality of microphones 12 are arrayed for listening to sounds within the body. The microphones 12 include outputs which couple to phase shifters. In this embodiment, noise cancellers receive the outputs of the phase shifters which then process the signals, such as through summing. In the event that this processing is performed in the analog domain, the output of the noise canceller is supplied to an analog to digital converter, whose output in turn is provided to the wireless transmission circuitry. An intelligent and cognitive system, depending on the usage scenario, is formed where all or part of the microphones already existing in the array reshape the beam for different applications. Hence, as the elements receive the signals, the output of the certain set of elements is utilized and fed to the signal processor to create an intelligent beam-forming system. The entire three-dimensional space is scanned as desired and depending on the application.

FIG. 8 shows a schematic block diagram of the functionalities of the system. The structures of FIGS. 6 A and D are shown for reference. The microphones 80 are arrayed to couple to the patient (optionally through buffer structures, shown in FIG. 7B). Substrate 84 holds the microphones 80, and optional sensor(s) 82. Communication paths 86 couple the signals for processing within the system. Any manner of communication path 86, whether wires, traces, vias, busses, wireless communication, or otherwise, may be utilized consistent with achieving the functionalities described herein. The communication paths 86 also function to provide command and control functions to the device components.

Broadly, the functionality may be classified into a conditioning module 90, a processing module 100 and a communication module 112, under control of a control system 120 and optionally a target selection module 122. The conditioning module 90 optionally includes an amplifier 92, filtering 94, and an analog to digital (ADC) converter 96. The processing module 100 optionally includes digital signal processor (DSP) 102, if processing is in the digital domain. Beam steering 104 and virtual focusing functionality 106 may optionally be provided. Noise cancellation 108 is preferably provided. Additional physical structures, such as a noise suppression screen may be supplied on the side of the device that is oriented to ambient noise in operation. De-convolver 110 serves to de-convolve the multiple sounds received from the body. The de-convolution may de-convolve heart sounds from lung sounds, or GI sounds. Sounds from a particular organ, e.g., the heart, may be even further de-convolved, such as into the well know cardiac sounds, including but not limited to first beat (S1), second beat (S2), sounds associated with the various valves, including the mitral, tricuspid, aortic and pulmonic valves, as well as to detect various conditions, such as heart murmur.

With intelligent scanning beam and appropriate selection of the number and placement of microphones in an array, the auscultation piece is placed in a single location and captures multiple sounds of interest (e.g., all the components of the heart and lung sounds), rather than moving the piece regularly as is the case in prior art systems. Further, the need for multiple auscultation pieces is eliminated as the beam electronically scans a range of angles, in addition to the normal angle.

FIG. 9 shows the device array based auscultation device 130 (as described in connection with the foregoing figures), as may communicate via wireless systems to various systems. The device 130 may communicate locally, such as to a wireless hearing piece 132. The wireless hearing piece 132 may be worn by the user, physician or other health care provider. The device may communicate with a personal communication device 134, e.g., PDA, cell phone, graphics enabled display, tablet computer, or the like, or with a computer 136. The device may communicate with a hospital server 138 or other medical data storage system. The data communicated may be acted up either locally or remotely by health care professionals, or in an automated system, to take the appropriate steps medically necessary for the user of the device 130.

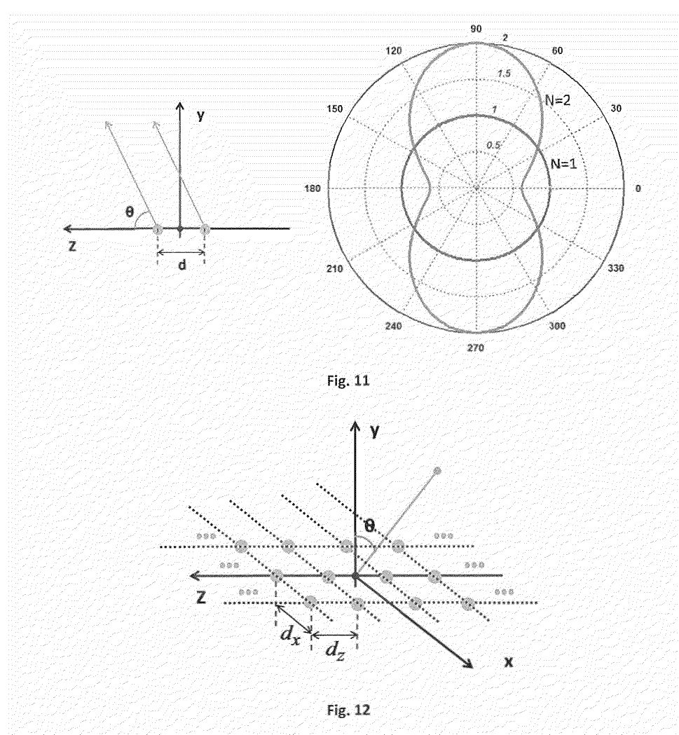

A common problem with current electronic stethoscopes is the noise levels and reverberations which require multiple filtering and signal processing, during which process part of the real signal might be removed as well. Increasing the directionality when capturing the signal leads to better quality sound recording; it also requires less processing and therefore less power consumption. In order to increase the directivity of a microphone, a larger diaphragm is optionally used, but there is a limit on enlarging the diaphragm. An alternative to enlarging the size of the auscultation element, without increasing the actual size of the microphone, is to assemble a set of smaller elements in an electrical and geometrical configuration. With a microphone array that is comprised of two or more MEMS microphones, the directionality of the microphone is increased, and specific nulls in desired spatial locations are created in order to receive a crisp and noise-free specific sound output. FIG. 11 shows the results of simulations for a two-element linear microphone array demonstrating the increase in the directivity and gain (along the desired direction) as compared to a single microphone. The circular display is for N=1, and the multi-modal display is for N=2. The angle convention is defined by FIG. 10.

Ultra miniature, e.g., 2 mm or less, and low power MEMS microphones with sensitivity of about 45-50 dB may be used. The device is optionally be implemented in a linear or planar array of two or more microphones for increased directivity and gain, as well as rejecting ambient noise; electronic steering of the directionality and virtual focusing are also enabled. FIG. 12 shows the architecture of an array where each microphone is shown as a point source on the grid. An advantage of using multiple microphones to capture sound is to allow further processing of the multiple sound signals to focus the receiving signal in the exact direction of the sound source. This processing is optionally accomplished by comparing the arrival times of the sound to each of the microphones. Then by providing effective electronic delay, and amplitude gain during the processing, the signals add constructively (i.e., add up) in the desired direction and destructively (i.e., cancel each other) in other directions. The higher directivity of the microphone array reduces the amount of captured ambient noise and reverberated sound.

The array may be formed in any manner or shape as to achieve the desired function of processing the sounds from the body. The array is optionally in the form of a grid. The grid may be a linear grid, or a non-linear grid. The grid may be a planar array, such as a n×n array. Optionally, the array may be a circular array, with or without a central microphone. The array may be a three-dimensional array. The separation between microphones may be uniform or non-uniform. The spacing between pairs of microphones may be 8 mm or less, or 6 mm or less, or 4 mm or less. The overall size of array is less than 3 square inches or less than 2 square inches, or less than a square inch. In one aspect, the minimum spacing between at least one pair of microphones is at least 2 centimeters, or at least 2.5 centimeters, or at least 3 centimeters.

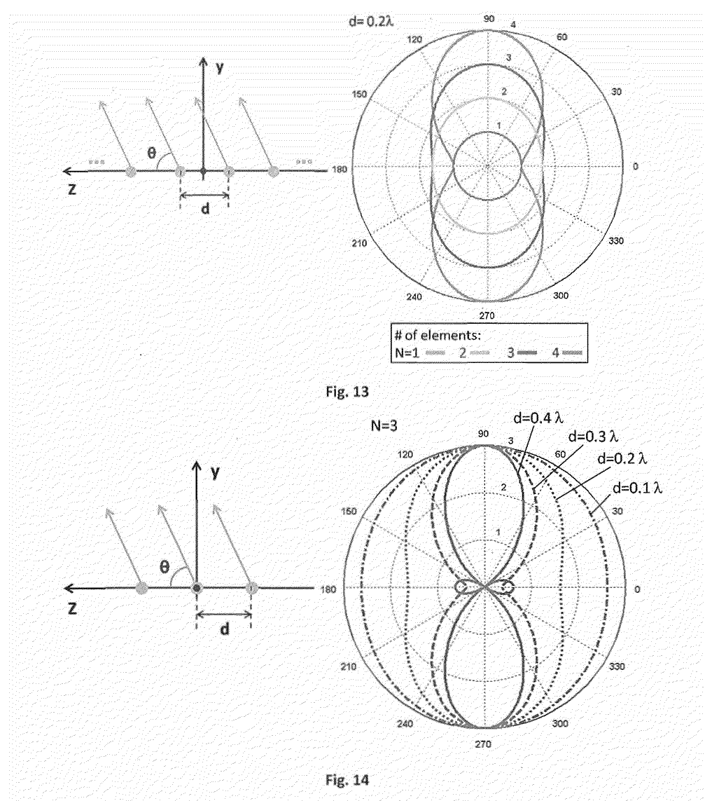

A linear array is composed of single microphone elements along a straight line (z-axis). As shown in FIG. 13, the gain and directivity of a microphone array improves as the size of the array grows. However, the power consumption and dimension of the processing unit sets a trade off in choosing the required number of array elements, linear or non-linearity of the array by choosing various spacing between elements, as does cost. As shown in FIG. 14, the geometrical placement of the elements plays a critical role in the response of the array, especially when scanning the beam by a constant gain and applying a progressive phase shift to each element. λ is the wavelength of the signal and is given by:

$$λ = \frac{v}{f} \qquad ( 1 )$$

where ν is the velocity of traveling wave and f represents the modulation frequency. The velocity of the sound in human's soft tissue is about 1540 msec, and the audible signal covers a bandwidth of 20 Hz to 2 KHz. Modulating this signal with a sampling frequency results in wavelengths in the range of a few inches. Preferably, there is at least one pair of microphones that are separated by 2.0 centimeters, and more preferably by 3 centimeters. In order to prevent frequency aliasing the elements of an array should be separated by a distance d, with the restriction being [5]:

$$d < \frac{λ}{2} \qquad ( 2 )$$

Hence, a separation of within a few millimeters is expected to form an effective array for listening to the body sounds. The scanning performance of a three-element array is shown in FIG. 15 for 0°, 30°, 45° and 60° progressive electronic phase shift, φ, between the elements to steer the beam accordingly.

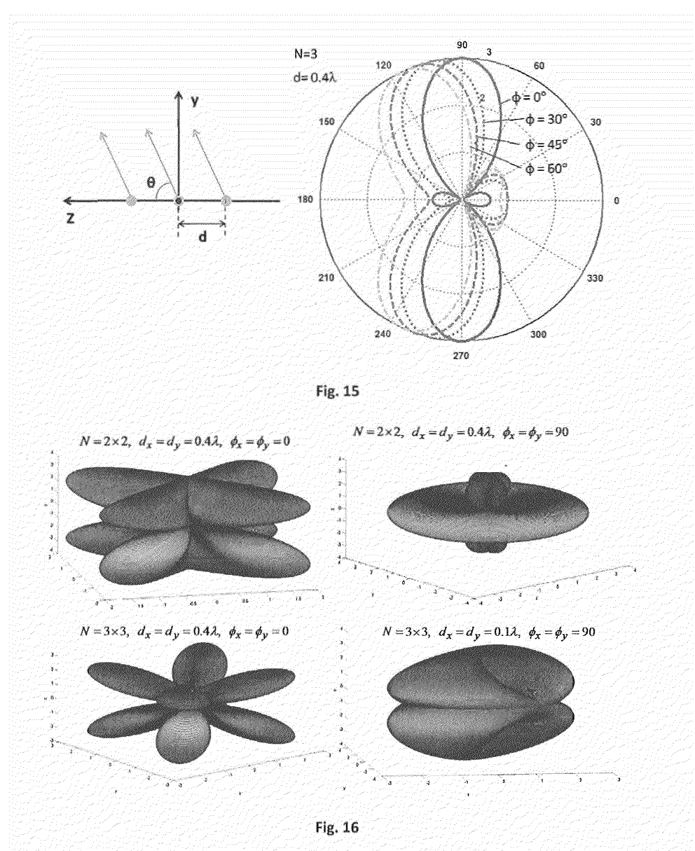

Increasing the number of elements in a planar fashion generates additional opportunities in creating nulls and maxima in the beam pattern of the array. FIG. 16 shows multiple different arrangements of microphones and underlines the importance of the design based on application considerations. The configuration of the array and location of the elements is fixed when the design is finalized based on the application considerations. Sound absorbing layers are optionally placed on the backside of the device to relinquish the signals from the back when necessary. The number of the elements to be utilized and their respective phase shift is programmed as desired.

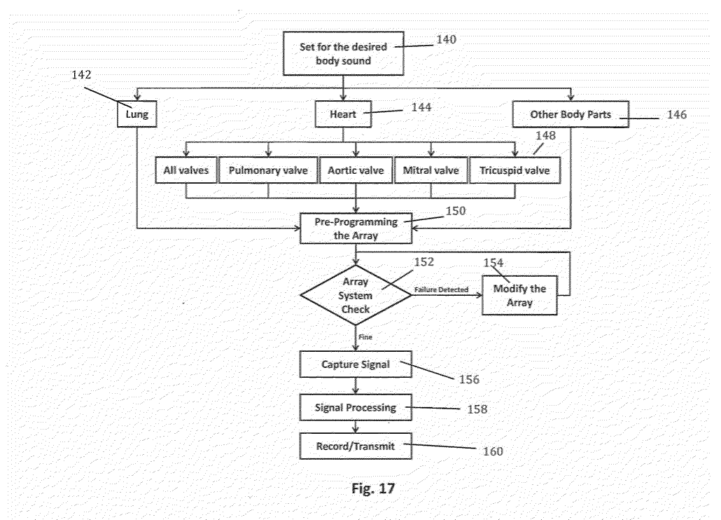

Finally, FIG. 17 provides a flowchart of an example of an operational process flow to capture the sound from a body organ of interest using the microphone array. Initially, the system is set for the desired body sound (step 140). This may be set locally by either the device user or by the medical care professional, such as through operation of the auscultation device 46 (FIG. 6A). Alternatively, the device may include a standard diagnostic program which will cycle between various sounds, or may include an intelligent selection program to set the device to detect the desired body sound. Alternately, a command may be sent from remote of the device to instruct the device as to the sounds to capture. As shown in FIG. 17, the sounds may include, by way of example, lung sounds 142, heart sounds 144 or other body part sounds 146, such as GI sounds. Optionally various sub-structures and their associated sounds (see, e.g., heart sounds 148) may be monitored. The array is pre-programmed at step 152. If a failure is detected, the array is modified at step 154. If there is no failure detected, the signal is captured at step 156, the signal processed at step 158 and optionally recorded and transmitted at step 160.

In order to further assist the user, colorful lights or LEDs (Red: weak signal level, Yellow: medium-to-moderate signal, and Green: strong signal level) are optionally incorporated into the auscultation piece to indicate when the user has placed it optimally, i.e., where the desired signal levels are strong. This is done by steering the gaze of the array and finding the direction where the signal levels are the strongest, or possess some other property, such as a recognizable sound from a particular body organ or portion of the body organ. Additional algorithms in connection with the captured signals may be used to guide the positioning for a specific recording, i.e., artificial intelligence capture of the skills of an experienced cardiologist in positioning of the piece and understanding the captured sounds. Various events may trigger the system to monitor for specific sounds. For example, if a pacemaker or other implanted device changes mode or take some action, the sensor may be triggered to search for and capture specific sounds.

Further elaboration of this technology is integration of additional ultra miniature and very low cost sensors into the platform for expanded diagnostic capabilities. A temperature sensor may optionally be included. In a wearable, adhesive patch, one or more accelerometers additionally capture the heart and respiration rate from the movement of the chest and monitor the activity level of the person. Optionally, other sensors include piezoelectric sensors, gyroscopes and ECG electrodes.

An added advantage of a microphone array is redundancy, i.e., the auscultation piece functions even if a microphone in the array malfunctions or fails. In this case, the problem microphone is disregarded in analyzing the signals.

All publications and patents cited in this specification are herein incorporated by reference as if each individual publication or patent application were specifically and individually indicated to be incorporated by reference. Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the following claims.

We claim:

1. An electronic device for receiving sounds in a body, comprising:
a plurality of microphones,
a plurality of buffer structures,
a patch structure, the patch structure including at least a patient side surface and an opposed side surface, the patch including a plurality of cavities, the cavities being adapted to receive the buffer structures and to maintain the buffer structures adjacent the plurality of microphones, at least two of the plurality of microphones being spaced at least 2 centimeters apart, and
device electronics, the device electronics including:
signal processing circuitry to analyze the sounds in the body, and
wireless transmission circuitry for sending information relating to the sounds in the body.

2. The electronic device of claim 1 wherein the buffer structure is rubber.

3. The electronic device of claim 1 wherein the buffer structure is metal.

4. The electronic device of claim 1 wherein the device includes adhesive to adhere the device to the body.

5. The electronic device of claim 1 wherein the chambers are 2 mm or less across.

6. The electronic device of claim 1 wherein the chambers are 3 mm or less across.

7. The electronic device of claim 1 wherein the device includes a directional processing system.

8. The electronic device of claim 1 wherein the device electronics de-convolve sounds in the body.

9. The electronic device of claim 1 wherein the device includes a noise cancellation system.

10. The electronic device of claim 1 wherein the device includes target selection circuitry.

11. An electronic scope for receiving sounds in a body, comprising:
a microphone array structure, the structure including at least:
a first microphone, the first microphone including an electrical output corresponding to sounds in the body,
a second microphone, the second microphone including an electrical output corresponding to sounds in the body, and
a support, the support being connected to at least the first and second microphones to hold them in an array configuration,
an analysis system, the analysis system including at least:
a directional processing system coupled to receive the output from the microphone array system, and
signal processing circuitry to analyze the sounds in the body, and
wireless transmission circuitry for sending information relating to the sounds in the body.

12. The electronic scope of claim 11 wherein the scope is a wearable patch.

13. The electronic scope of claim 11 wherein the array is a planar array.

14. The electronic scope of claim 11 wherein the array is a three-dimensional array.

15. The electronic scope of claim 11 wherein the microphones are MEMS microphones.

16. The electronic scope of claim 11 wherein the microphones are piezoelectric sensors.

17. The electronic scope of claim 11 wherein the distance between at least two microphones in the array is 2 centimeters.

18. The electronic scope of claim 11 further including target selection circuity.

19. An electronic scope for receiving sounds in a body, comprising:
a microphone array structure, the array including at least:
a first microphone, the first microphone including an electrical output corresponding to sounds in the body,
a second microphone, the second microphone including an electrical output corresponding to sounds in the body, and
a support, the support being connected to at least the first and second microphones to hold them in an array configuration,
an analysis system, the system including at least:
inputs adapted to receive the at least first and second signals corresponding to body sounds, and
digital processing circuitry to filter, amplify and combine the signals to provide for electronic spatial scanning of the body.

20. The electronic scope of claim 19 wherein the analysis system de-convolves the sounds of the body.
